(19) United States
(12) Patent Application Publication
(10) Pub. No.: US 2010/0209965 A1
(43) Pub. Date: Aug. 19, 2010

(54) CATALYTIC PYROLYSIS OF FINE PARTICULATE BIOMASS, AND METHOD FOR REDUCING THE PARTICLE SIZE OF SOLID BIOMASS PARTICLES

(75) Inventors: Paul O'Connor, Hoevelaken (NL); Dennis Stamires, Dana Point, CA (US)

Correspondence Address: Kior Inc., 13001 Bay Park Rd., Pasadena, TX, 77507, US

(73) Assignees: BIOECON INTERNATIONAL HOLDING N.V., CURACAO (NL); Kior Inc.

(21) Appl. No.: 12/373,748

(22) PCT Filed: Jul. 13, 2007
(86) PCT No.: PCT/EP2007/057269

§ 371 Date: Jan. 14, 2009

(30) Foreign Application Priority Data
Jul. 17, 2006 (US) 60831242

(51) Int. Cl. C10B 57/00; C12P 1/00; C09K 3/00

(52) U.S. Cl. 435/41; 201/2.5; 252/182.11

(57) ABSTRACT

A process is disclosed for converting a particulate biomass material to a bioliquid. In the process the biomass material is mixed with a heat transfer medium and a catalytic material, and heated to a temperature in the range of from 150 to 600° C. The particle size of the solid biomass may be reduced by abrasion in admixture with inorganic particles under agitation by a gas. The biomass particles of reduced size obtained in the abrasion process may be converted to bioliquid in any of a number of conversion processes.

### BACKGROUND OF THE INVENTION

The present invention relates to an improved process for the thermal conversion of a particulate carbon based energy source, in particular fine particulate biomass.

One of the challenges in the thermal conversion of solid biomass is to provide a suitable medium for transferring heat energy to the particulate material. Sand has been proposed as such a suitable medium, and the use of sand in a fluidized bed process for the thermal conversion of biomass has been reported. However, sand is intrinsically inert and does not contribute to the thermal conversion reaction itself, other than in its role as a heat transfer medium.

Another one of the challenges in the thermal conversion of solid biomass is to provide the biomass in a particle size that is conducive to such thermal conversion.

It is an object of the present invention to modify a heat transfer medium, such as sand, so as to provide it with catalytic properties. Specifically, it is an object of the present invention to impart to a heat transfer medium, such as sand, catalytic properties that are conducive to thermally converting solid particulate biomass under relatively mild reaction conditions.

It is a further object of the present invention to provide a process for reducing the particle size of a solid biomass material.

### BRIEF SUMMARY OF THE INVENTION

The present invention relates to a process for the thermal conversion of a fine solid particulate biomass comprising the steps of providing a mixture of the solid particulate biomass, a heat transfer medium, and a catalytically active material; heating the mixture to a temperature of from 150 to 600° C.

The heat transfer medium preferably is an inorganic particulate material.

In a preferred embodiment of this invention the fine solid particulate biomass is prepared by fluid abrasion of a solid particulate biomass in the presence of the inert particulate inorganic material.

### DESCRIPTION OF ILLUSTRATIVE EMBODIMENTS

The present invention relates to a process for the thermal conversion of solid particulate biomass. As used herein, the term particulate material refers to materials that are solid and in a finely divided form. An example includes biomass in a finely divided form, such as saw dust or ground straw.

In prior art processes, biomass particles are mixed with sand in a thermal conversion process, such as a fluidized bed process. In these processes, sand acts as a carrier for transferring heat energy to the biomass material, and also as a sink for tar that is produced during the thermal conversion process.

Being an inert material, sand does not contribute to the thermal conversion process itself. A draw-back of the prior art processes is that they require a relatively high conversion temperature. Consequently, the prior art thermal conversion processes require a large input of heat energy. In addition, the high conversion temperature results in excessive cracking of the carbon based energy source material, associated with the formation of significant quantities of tar. It is, therefore, desirable to develop a process permitting the thermal conversion of a carbon based energy source at a lower temperature than is possible in the prior art processes.

It has been found that the thermal conversion of biomass materials may be carried out at milder conditions of temperature if the process is carried out in the presence of both a heat transfer medium, for example an inert particulate inorganic material, and a catalytically active material.

In a specific embodiment the particulate inorganic material is used that is both a heat transfer medium and a catalyst.

In a specific embodiment, the catalytically active material is an inorganic oxide in particulate form. Preferably, the particulate inorganic oxide is selected from the group consisting of refractory oxides, clays, hydrotalcites, crystalline aluminosilicates, layered hydroxyl salts, and mixtures thereof.

Examples of refractory inorganic oxides include alumina, silica, silica-alumina, titania, zirconia, and the like. Refractory oxides having a high specific surface are preferred. Specifically, preferred materials have a specific surface area as determined by the Brunauer Emmett Teller (“BET”) method of at least 50 m²/g.

Suitable clay materials include both cationic and anionic clays. Suitable examples include smectite, bentonite, sepiolite, atapulgite, and hydrotalcite.

Other suitable metal hydroxides and metal oxides include bauxite, gibbsite and their transition forms. Cheap catalytic material may be lime, brine and/or bauxite dissolved in a base (NaOH), or natural clays dissolved in an acid or a base, or fine powder cement from a kiln.

The term “hydrotalcites” as used herein include hydrotalcite per se, as well as other mixed metal oxides and hydroxides having a hydrotalcite-like structure, as well as metal hydroxyl salts.

The catalytically active material may comprise a catalytic metal. The catalytic metal may be used in addition to or in lieu of the catalytically active inorganic oxide. The metal may be used in its metallic form, in the form of an oxide, hydroxide, hydroxyl oxide, a salt, or as a metallo-organic compound, as well as materials comprising rare earth metals (e.g. bastnesite).

Preferably, the catalytic metal is a transition metal, more preferably a non-noble transition metal. Specifically preferred transition metals include iron, zinc, copper, nickel, and manganese, with iron being the most preferred.

There are several ways in which the catalytic metal compound can be introduced into the reaction mixture. For example, the catalyst may be added in its metallic form, in the form of small particles. Alternatively, the catalyst may be added in the form of an oxide, hydroxide, or a salt. In one preferred embodiment, a water-soluble salt of the metal is mixed with the carbon based energy source and the inert particulate inorganic material in the form of an aqueous slurry. In this particular embodiment, it may be desirable to mix the particles of the biomass with the aqueous solution of the metal salt before adding the inert particulate inorganic material, so as to make sure that the metal impregnates the biomass material. It is also possible to first mix the biomass with the inert particulate inorganic material, prior to adding the aqueous solution of the metal salt. In yet another embodiment, the aqueous solution of the metal salt is the first mixed with the particulate inert inorganic material, whereupon the material is dried prior to mixing it with the particulate biomass In this embodiment, the inert inorganic particles are converted to heterogeneous catalyst particles.

The specific nature of the inert particulate inorganic material is not of critical importance for the process of the present invention, as its main function is to serve as a vehicle for heat transfer. Its selection will in most cases be based on considerations of availability and cost. Suitable examples include quartz, sand, volcanic ash, virgin (that is, unused) inorganic sandblasting grit, and the like. Mixtures of these materials are also suitable. Virgin sandblasting grit is likely to be more expensive than materials such as sand, but it has the advantage of being available in specific ranges of particle size and hardness.

When used in a fluidized bed process, the inert particulate inorganic material will cause a certain level of abrasion of the walls of the reactor, which is typically made of steel. Abrasion is generally undesirable, as it causes an unacceptable reduction in the useful life of the reactor. In the context of the present invention, a moderate amount of abrasion may in fact be desirable. In case there is abrasion, such abrasion could introduce small particles of metal into the reaction mixture, comprising the metal components of the steel of the reactor (mainly Fe, with minor amounts of, for example, Cr, Ni, Mn, etc.). This could impart a certain amount of catalytic activity to the inert particulate inorganic material. It will be understood that the term “inert particulate inorganic material” as used herein includes materials that are by their nature inert, but have acquired a certain degree of catalytic activity as a result of having been contacted with, for example metal compounds.

Sandblasting grit that has previously been used in a sandblasting process is particularly suitable for use in the process of the present invention. Used sandblasting grit is considered a waste material, which is abundantly available at a low cost. Preferred are sandblasting grit materials that have been used in the sandblasting of metal surfaces. During the sandblasting process the grit becomes intimately mixed with minute particles of the metal being sandblasted. In many cases the sandblasted metal is steel. Grit that has been used in the sandblasting of steel presents an intimate mixture comprising small particles of iron, and lesser quantities of other suitable metals such as nickel, zinc, chromium, manganese, and the like. Being in essence a waste product, grit from a sandblasting process is abundantly available at a low cost. Nevertheless, it is a highly valuable material in the context of the process of the present invention.

The effective contacting of the carbon based energy source, the inert inorganic material and the catalytic material is essential and can proceed via various routes. The two preferred routes are:

The dry route, whereby a mixture of the particulate biomass material and the inert inorganic material is heated and fluidized, and the catalytic material is added as fine solid particles to this mixture.

The wet route, whereby the catalytic material is dispersed in a solvent and this solvent is added to the mixture of particulate biomass material and the inert inorganic material. A preferred solvent is water.

The term “fine particulate biomass” as used herein refers to biomass material having a mean particle size in the range of from 0.1 mm to 3 mm, preferably from 0.1 mm to 1 mm.

Biomass from sources such as straw and wood may be converted to a particle size in the range of 5 mm to 5 cm with relative ease, using techniques such as milling or grinding. For an effective thermal conversion it is desirable to further reduce the mean particle size of the biomass to less than 3 mm, preferably less than 1 mm. Comminuting biomass to this particle size range is notoriously difficult. It has now been discovered that solid biomass may be reduced in particle size to a mean particle size range of from 0.1 mm to 3 mm by abrading biomass particles having a mean particle size in the range of 5 mm to 50 mm in a process involving mechanical mixing of the biomass particles with an inorganic particulate material and a gas.

Abrasion of particles in a fluid bed process is a known, and in most contexts an undesirable phenomenon. In the present context this phenomenon is used to advantage for the purpose of reducing the particle size of solid biomass material.

Thus, in one embodiment of the present invention, biomass particles having a particle size in the range of from 5 mm to 50 mm are mixed with inorganic particles having a particle size in the range of from 0.05 mm to 5 mm. This particulate mixture is agitated with a gas. As the inorganic particles have a hardness that is greater than that of the biomass particles, the agitation results in a reduction of the size of the biomass particles. Suitably this process is used for reducing the particle size of the biomass to 0.1 to 3 mm.

The amount of agitation of the particulate mixture determines to a large extent the rate of size reduction of the biomass particles. In order of increasing abrasion activity, the agitation may be such as to form a fluid bed, a bubbling or ebullient bed, a spouting bed, or pneumatic conveyance. For the purpose of the present invention, spouting beds and pneumatic conveyance are the preferred levels of agitation.

The gas may be air, or may be a gas having a reduced level of oxygen (as compared to air), or may be substantially oxygen-free. Examples include steam, nitrogen, and gas mixtures as may be obtained in a subsequent thermal conversion of the fine biomass particles. Such gas mixtures may comprise carbon monoxide, steam, and/or carbon dioxide.

The abrasion process may be carried out at ambient temperature, or at an elevated temperature. The use of elevated temperatures is preferred for biomass particles containing significant amounts of moisture, because it results in a degree of drying of the biomass particles. Drying increases the hardness of the biomass particles, making the particles more susceptible to size reduction by abrasion. Preferred drying temperatures range from about 50 to 150° C. Higher temperatures are possible, in particular if the agitating gas is oxygen-poor or substantially oxygen-free.

Preferred for use in the abrasion process are those inorganic particles that will be used in a subsequent thermal conversion process according to the present invention. In a still further preferred embodiment the catalytic material is also present during the abrasion process. It is believed that some of the catalytic material, if present during the abrasion process, becomes embedded in the biomass particles, which makes the subsequent thermal conversion process more effective.

In a particularly preferred embodiment of the present invention, biomass particles having a particle size in the range of 5 mm to 50 mm are mixed with inert inorganic particles and a catalytic material. This mixture is agitated by a gas, preferably resulting in the formation of a spouting bed or pneumatic conveyance. After the biomass particles reach a mean particle size in the range of 0.1 mm to 3 mm the temperature is increased to 150 to 600° C.

The small biomass particles obtained in the abrasion process are particularly suitable for conversion to a bioliquid in a suitable conversion process. Examples of suitable conversion processes include hydrothermal conversion, enzymatic conversion, pyrolysis, catalytic conversion, and mild thermal conversion.

A specific aspect of the present invention is a process for preparing a bioliquid from a solid biomass material, said process comprising the steps of:
a) providing the solid biomass in the from of particles having a particle size of greater than 5 mm;
b) mixing the biomass particles of step a) with an inorganic particulate material having a particle size in the range of 0.05 mm to 5 mm;
c) agitating the mixture obtained in step b) with a gas whereby the particle size of the biomass is reduced to 0.1 to 3 mm;
d) subjecting the biomass particles obtained in step c) to hydrothermal conversion.

Another specific aspect of the present invention is a process for preparing a bioliquid from a solid biomass material, said process comprising the steps of:
a) providing the solid biomass in the from of particles having a particle size of greater than 5 mm;
b) mixing the biomass particles of step a) with an inorganic particulate material having a particle size in the range of 0.05 mm to 5 mm;
c) agitating the mixture obtained in step b) with a gas whereby the particle size of the biomass is reduced to 0.1 to 3 mm;
d) subjecting the biomass particles obtained in step c) to an enzymatic conversion.

Yet another specific aspect of the present invention is a process for preparing a bioliquid from a solid biomass material, said process comprising the steps of:
a) providing the solid biomass in the from of particles having a particle size of greater than 5 mm;
b) mixing the biomass particles of step a) with an inorganic particulate material having a particle size in the range of 0.05 mm to 5 mm;
c) agitating the mixture obtained in step b) with a gas whereby the particle size of the biomass is reduced to 0.1 to 3 mm;
d) subjecting the biomass particles obtained in step c) to catalytic conversion.

Yet another specific embodiment of the present invention is a process for preparing a bioliquid from a solid biomass material, said process comprising the steps of:
a) providing the solid biomass in the from of particles having a particle size of greater than 5 mm;
b) mixing the biomass particles of step a) with an inorganic particulate material having a particle size in the range of 0.05 mm to 5 mm;
c) agitating the mixture obtained in step b) with a gas whereby the particle size of the biomass is reduced to 0.1 to 3 mm;
d) subjecting the biomass particles obtained in step c) to a hydrothermal conversion.

In another embodiment the invention relates to a process for preparing a bioliquid from a solid biomass material, said process comprising the steps of:
a) providing the solid biomass in the from of particles having a particle size of greater than 5 mm;
b) mixing the biomass particles of step a) with an inorganic particulate material having a particle size in the range of 0.05 mm to 5 mm;
c) agitating the mixture obtained in step b) with a gas whereby the particle size of the biomass is reduced to 0.1 to 3 mm;
d) subjecting the biomass particles obtained in step c) to catalytic conversion.

Preferably, step d) is performed in a reductive atmosphere e.g., a gas mixture comprising hydrogen and/or CO.

Yet another specific aspect of the present invention is a process for preparing a bioliquid from a solid biomass material, said process comprising the steps of:
a) providing the solid biomass in the from of particles having a particle size of greater than 5 mm;
b) mixing the biomass particles of step a) with an inorganic particulate material having a particle size in the range of 0.05 mm to 5 mm;
c) agitating the mixture obtained in step b) with a gas whereby the particle size of the biomass is reduced to 0.1 to 3 mm;
d) subjecting the biomass particles obtained in step c) to mild thermal conversion.

The thermal conversion may be performed in the presence of hydrogen.

The thermal conversion process may be carried out under atmospheric pressure, or under reduced pressure, reduced pressure being preferred. The thermal conversion is preferably carried out in an oxygen-poor or, more preferably, an oxygen-free atmosphere.

In a particularly preferred embodiment the thermal conversion is carried out in a fluid bed reactor, for example the type of reactor commonly used in fluid catalytic cracking of crude oil fractions. The temperature in the reactor may be uniform, or the reactor may be operated such that zones of different temperatures are established within the reactor. Advantageously two or more temperature zones may exist within the reactor, with the lowermost zone having the lowest temperature, and the temperature of each zone being higher than that of the zone immediately below it.

The thermal conversion may be carried out in a single reactor, or in a series of two or more reactors. If more than one reactor is used, it is advantageous to operate the individual reactors under different reaction conditions. Examples of reaction conditions include pressure, temperature, and/or fluidization state.

During the thermal conversion a carbon deposit, e.g. in the form of tar or coke, may form on the particulate heat transfer medium and the particulate catalytic material. In a preferred embodiment the carbon deposit is burned off, and the heat generated in the burning off process may be used for keeping the reactor at the desired temperature. After the hat transfer medium and the catalytic material have been regenerated in this fashion they can suitably be re-introduced into the reactor. Optionally catalytic material may be replenished before this re-introduction into the reactor.

Thus, the invention has been described by reference to certain embodiments discussed above. It will be recognized that these embodiments are susceptible to various modifications and alternative forms well known to those of skill in the art.

Many modifications in addition to those described above may be made to the structures and techniques described herein without departing from the spirit and scope of the invention. Accordingly, although specific embodiments have been described, these are examples only and are not limiting upon the scope of the invention.

What is claimed is:

1. A process for the thermal conversion of a fine particulate biomass comprising the steps of:
a) providing a mixture of the fine particulate biomass, a heat transfer medium, and a catalytic material;
b) heating said mixture to a temperature of from 150 to 600° C.

2. The process of claim 1 wherein the heat transfer medium is sand.

3. The process of claim 1 wherein the catalytic material is in a particulate form.

4. The process of claim 1 wherein the catalytic material comprises a transition metal.

5. The process of claim 4 wherein the catalytic metal is a non-noble transition metal.

6. The process of claim 5 wherein the catalytic metal is selected from the group consisting of Fe, Zn, Mn, Cu, Ni and mixtures thereof.

7. The process of claim 1 wherein the catalytic material is an inorganic oxide or an inorganic hydroxide.

8. The process of any one of the preceding claims wherein step a) comprises
the sub-steps of mixing biomass particles having a particle size in the range of 5 to 50 mm with an inorganic particulate material having a particle size in the range of 0.05 mm to 5 mm, and agitating the mixture with a gas whereby the particle size of the biomass is reduced to 0.1 to 3 mm.

9. The process of claim 8 wherein the particulate mixture further comprises a catalytic material.

10. The process of claim 8 wherein the inorganic particulate material has catalytic activity.

11. The process of any one of claims 8 to 10 wherein the agitating gas is air.

12. The process of any one of claims 8 to 10 wherein the agitating gas is oxygen-poor.

13. The process of claim 12 wherein the agitating gas is substantially oxygen-free.

14. The process of any one of claims 8 to 13 wherein the particulate mixture is agitated to form a fluid bed, an ebullient bed, or a spouting bed.

15. The process of any one of claims 8 to 13 wherein the particulate mixture is agitated to the point of pneumatic conveyance.

16. The process of any one of claims 8 to 15 wherein the particulate mixture is agitated at a temperature in the range of 50 to 150° C.

17. The process of claim 1 wherein step a) comprises:
mixing particulate biomass material and an inert inorganic material;
heating and fluidizing the mixture;
adding catalytic material to the fluidized mixture in the form of fine solid particles.

18. The process of claim 1 wherein step a) comprises:
dispersing the catalytic material in a solvent;
providing a mixture of particulate biomass material and particulate inert inorganic material;
adding the dispersed catalytic material to said mixture.

19. The process of claim 2 wherein the heat transfer medium is sand that has been used in a sandblasting process.

20. The process of claim 19 wherein the sand has been used in the sandblasting of steel.

21. The process of claim 1 carried out in a reactor which is operated under reduced pressure.

23. The process of claim 1 carried out in a reactor which is operated in an oxygen-poor atmosphere.

24. The process of claim 1 carried out in a reactor containing more than one temperature zone.

25. The process of claim 1 carried out in more than one reactor, each operating under different reaction conditions.

26. The process of claim 1 comprising the further step of removing a carbon deposit from the heat transfer medium by burning, and using heat resulting from this burning in the thermal conversion process.

27. A process for preparing a bioliquid from a solid biomass material, said process comprising the steps of:
a) providing the solid biomass in the from of particles having a particle size of greater than 5 mm;
b) mixing the biomass particles of step a) with an inorganic particulate material having a particle size in the range of 0.05 mm to 5 mm;
c) agitating the mixture obtained in step b) with a gas whereby the particle size of the biomass is reduced to 0.1 to 3 mm;
d) subjecting the biomass particles obtained in step c) to hydrothermal conversion.

28. A process for preparing a bioliquid from a solid biomass material, said process comprising the steps of:
a) providing the solid biomass in the from of particles having a particle size of greater than 5 mm;
b) mixing the biomass particles of step a) with an inorganic particulate material having a particle size in the range of 0.05 mm to 5 mm;
c) agitating the mixture obtained in step b) with a gas whereby the particle size of the biomass is reduced to 0.1 to 3 mm;
d) subjecting the biomass particles obtained in step c) to enzymatic conversion.

29. A process for preparing a bioliquid from a solid biomass material, said process comprising the steps of:
a) providing the solid biomass in the from of particles having a particle size of greater than 5 mm;
b) mixing the biomass particles of step a) with an inorganic particulate material having a particle size in the range of 0.05 mm to 5 mm;
c) agitating the mixture obtained in step b) with a gas whereby the particle size of the biomass is reduced to 0.1 to 3 mm;
d) subjecting the biomass particles obtained in step c) to thermal conversion.

30. A process for preparing a bioliquid from a solid biomass material, said process comprising the steps of:
a) providing the solid biomass in the from of particles having a particle size of greater than 5 mm;
b) mixing the biomass particles of step a) with an inorganic particulate material having a particle size in the range of 0.05 mm to 5 mm;
c) agitating the mixture obtained in step b) with a gas whereby the particle size of the biomass is reduced to 0.1 to 3 mm;
d) subjecting the biomass particles obtained in step c) to a hydrothermal conversion.

31. A process for preparing a bioliquid from a solid biomass material, said process comprising the steps of:
a) providing the solid biomass in the from of particles having a particle size of greater than 5 mm;
b) mixing the biomass particles of step a) with an inorganic particulate material having a particle size in the range of 0.05 mm to 5 mm;
c) agitating the mixture obtained in step b) with a gas whereby the particle size of the biomass is reduced to 0.1 to 3 mm;
d) subjecting the biomass particles obtained in step c) to catalytic conversion.

32. The process of claim 31, whereby step d) is performed in a reductive atmosphere.
